(19) United States
(12) Patent Application Publication
(10) Pub. No.: US 2020/0237305 A1
(43) Pub. Date: Jul. 30, 2020

(54) SENSOR POSITIONING FOR TRIAGING CARDIAC DATA

(71) Applicant: RCE Technologies, Inc., Decatur, GA (US)

(72) Inventors: Atandra Burman, Decatur, GA (US); Siddharth Biswal, Decatur, GA (US); Jitto Titus, Acworth, GA (US)

(21) Appl. No.: 16/774,348

(22) Filed: Jan. 28, 2020

Related U.S. Application Data
(60) Provisional application No. 62/797,905, filed on Jan. 28, 2019.

(51) Int. Cl. A61B 5/00; G16H 50/30; G16H 40/67; G16H 50/20; G16H 15/00; A61B 5/1455; A61B 5/0408

(52) U.S. Cl. CPC A61B 5/6804; G16H 50/30; G16H 40/67; G16H 50/20; A61B 5/6805; A61B 5/1455; A61B 5/04085; A61B 5/7264; G16H 15/00

(57) ABSTRACT

A method of monitoring cardiac health of a human (user) includes measuring back portion cardiac data using one or more sensors that are all in contact with a posterior of a human torso. The method further includes detecting a cardiac anomaly using machine learning based on the back portion cardiac data. In one or more examples, the method further includes converting the back portion cardiac data to front portion cardiac data using a translation model, wherein the front portion cardiac data is used for detecting the cardiac anomaly. In one or more examples, the one or more sensors are positioned using a panel that is incorporated into a garment that is in contact with the human torso. The panel can be detachable from the garment in one or more examples.

### PRIORITY

This application claims priority to the provisional patent application U.S. 62/797,905 that was filed on Jan. 28, 2019, contents of which are included herein in entirety.

### BACKGROUND

Every 40 seconds, someone in the United States has a heart attack, and 1 out of every 4th person has a repeat MI (Myocardial Infarction). There are globally around 210,000 repeat heart attacks per year, with an average time to intervention after reported pain being 75 minutes. 1 out of 5 heart attacks are silent, along with unmet needs of diabetic people with peripheral neuropathy who do not feel pain. Given 800,000 people in the US, and 34 million people worldwide suffer this unexpected event, healthcare needs an early triage solution in preventing these unexpected life changing events. While average hospitalization in such cases of critical heart events runs an approximate 20,000 US dollars, not all of this is reimbursed by Medicare, making the much-needed push for remote monitoring and preventive medicine, while keeping health care costs low.

### SUMMARY

According to one or more embodiments of the present invention, a system for monitoring cardiac health of a human (user) includes a garment. The garment includes a front portion that contacts the anterior of a human torso, and a back portion that contacts the posterior of the human torso. The back portion includes one or more cardiac sensors that are used to monitor the cardiac health of the user.

In one or more embodiments of the present invention, the garment includes a panel that is incorporated with the back portion, and wherein the panel includes the cardiac sensors. The panel is detachable from the back portion in one or more examples.

According to one or more embodiments of the present invention, a method of monitoring cardiac health of a human (user) includes measuring back portion cardiac data using one or more sensors that are all in contact with a posterior of a human torso. The method further includes detecting a cardiac anomaly using machine learning based on the back portion cardiac data. In one or more examples, the method further includes converting the back portion cardiac data to front portion cardiac data using a translation model, wherein the front portion cardiac data is used for detecting the cardiac anomaly. In one or more examples, the one or more sensors are positioned using a panel that is incorporated into a garment that is in contact with the human torso. The panel can be detachable from the garment in one or more examples.

The above-described features can also be provided at least by a system, a computer program product, and a machine, among other types of implementations.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a block diagram of an example system using a sensor positioning panel for triaging cardiac data according to one or more embodiments of the present invention;

FIG. 2 depicts an example panel according to one or more embodiments of the present invention;

FIG. 3 depicts a block diagram of an example garment according to one or more embodiments of the present invention;

FIG. 4 depicts a flowchart of an example method of using a sensor positioning panel for triaging cardiac data according to one or more embodiments of the present invention;

FIG. 5 depicts an example computer system that can implement one or more embodiments of the present invention; and

FIG. 6 depicts an example block diagram of a data analyzer according to one or more embodiments of the present invention.

The diagrams depicted herein are illustrative. There can be many variations to the diagrams or the operations described therein without departing from the spirit of the invention. For instance, the actions can be performed in a differing order or actions can be added, deleted or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Although we have seen technological advancements in cardiac care biomedical devices and connected software, technical challenges still exist regarding consistent, accurate and real-time data collection devices that interface with the biomedical devices. The technical solutions described herein address such technical challenges, thereby enabling an effective cardiology workflow which prevents critical heart events.

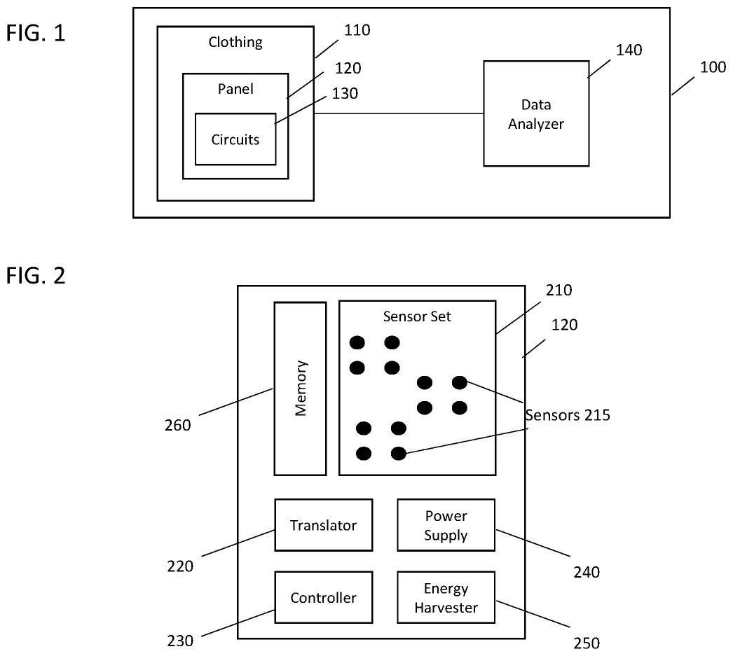

FIG. 1 depicts a block diagram of an example system using a sensor positioning panel for triaging cardiac data according to one or more embodiments. Components of the system 100 include, but are not limited to a clothing material 110, a panel 120 that is integrated with the clothing material 110, and circuits 120 that are integrated with the panel 120. The system 100 further includes a data analyzer 140. The system 100 provides, in one or more examples, a triage platform, where the components described herein together facilitate a triage workflow to analyze heart related sensor measurements obtained using the panel 120.

In one or more examples, the panel 120 can be an integrated circuit, such as an application specific integrated circuit (ASIC) that is manufactured on a flexible material, which can be bent or curved to fit one or more contours of a human body. For example, the panel 120 can be a fabric with the circuits 130 integrated using adhesive, stitches, clips, or any other technique. Alternatively, or in addition, the panel 120 can be a fabric with the circuits 130 integrated in the fabric itself, for example, in the weaving of the fabric, between two or more layers of the fabric, etc. In one or more examples, the circuits 130 are embedded into the panel 120 using knitting and transfer print technologies. Alternatively, or in addition, the panel 120 is manufactured using silicon, or other such base layer material, with the circuits “printed” on the base layer material using photolithography, or other such technology. Accordingly, the panel 120 is a system on a chip (SoC) that has flexible base layer that facilitates the panel 120 to be bent or curved to match contours of a human body.

The circuits 130 includes optical sensors, electrocardiogram (ECG) sensors, and other types of sensors. The optical sensors non-invasively detect protein biomarkers present in the blood of a human body that is in contact with the patch 120. Another embodiment of the optical sensors may include hemodynamic monitoring function. The ECG sensors provide a 3D representation of heart beat data of the user that is in contact with the patch 120.

The patch 120 can be incorporated into clothing 110, that can include wearable clothes, and for example are part of a garment, such as a shirt, pant, underwear, vests, sweaters, jackets, etc. Alternatively, or in addition, the patch 120 can be incorporated into other types of clothing material 110, such as bedding, (including but not limited to bedsheets and blankets), aprons, gowns, and other such clothes. In one or more embodiments of the present invention, the panel 120 is an integral part of the clothing 110 itself. For example, the panel 120 is a back of a shirt, under-garment, or any other wearable clothing item. Alternatively, in one or more embodiments of the present invention, the panel 120 is a detachable portion from the clothing item 110. For example, the panel 120 can be attached/detached from the clothing item 110 using adhesive, stitches, buttons, clips, or any other such technique.

By embedding the panel 120 into the clothing material 110, which can be used for conventional daily wear (garments) or other use of the clothing material 110 (sheets), the panel 120 provides heart health monitoring in a seamless manner.

FIG. 2 depicts an example patch according to one or more embodiments. The panel 120 includes a sensor-set 210, a translator 220, a controller 230, a power supply 240, an energy harvester 250, and a memory 260. In one or more embodiments of the present invention, the panel 120 can include additional components.

The panel 120 includes a sensor-set 210 that includes a predetermined number of sensors 215, such as 10 sensors, that pick up signals from the body that can be represented graphically for detecting any abnormality in heart function. The sensors 215 can include ECG sensors, optical sensors, position sensors, and other types of sensors that can facilitate in acquiring data that can be used to triage cardiac data.

The controller 230 includes one or more processing units that operate one or more predetermined functions associated with the panel 120. For example, the controller 230 can be responsible for periodically causing the sensors 215 from the sensor-set 210. Further, the controller 230 can be used to configure one or more settings of the sensor-set 210. For example, the parameters can include a schedule of when sensor data is captured by the sensor-set 210. Further, the parameters can include the type of measurements captured by the sensor-set 210. Further yet, the parameters can include how long the sensor data is stored on the panel 120. The sensor data that is captured is stored on the memory 260. Further, the controller 230 can facilitate communication of the sensor data to the data analyzer 140, in a wired or a wireless manner. The controller 230 can include communication modules for such data transfer.

The power supply 240 provides power to the controller 230, the sensor-set 210 and other components of the panel 120. The power supply 240 can be a battery pack. In one or more embodiments of the present invention, the energy harvester 250 generates power that can be stored in the power supply 240. The power is generated based on the user's movements, in one or more examples. Alternatively, or in addition, the energy harvester 250 charges the power supply 240 using electrical power received via an electrical port, such as a USB port or the like.

The sensors 215 are brought in contact with a human body of the user using electrodes. A technical challenge with these electrodes is ensuring contact of the electrodes with the body, particularly when the user is sleeping, in motion (such as during an activity), sitting, standing, and various other states.

In conventional cardiac triaging systems, sensors are placed so as to contact the front side (anterior) of the user's torso. The data acquired by the sensors in these positions are used by clinicians, and other medical personnel for triaging any conditions that the user may be experiencing/may have experienced. However, a technical challenge is ensuring that the sensors are in contact with the user to acquire precise measurements. Because of different body types, positioning the sensors in a clothing material, such as a wearable garment to ensure consistent contact with the user is a technical challenge, and that is addressed by the technical solutions described herein. Further, the technical solutions described herein facilitate such consistent contact even in case of movements/motion by the user, thus, not restricting the user when wearing/using the smart garment with embedded sensors.

The technical solutions described herein address such technical challenges by facilitating the panel 120 to be placed in a position where the sensor-set 210 is in consistent physical contact with user's body and particularly on the back of the user (posterior). Depending on the user demographic, elasticity can be introduced at strategic sections of the panel 120 to further improve the quality of electrode contact with the body. The strategic sections are predetermined portions of the panel 120.

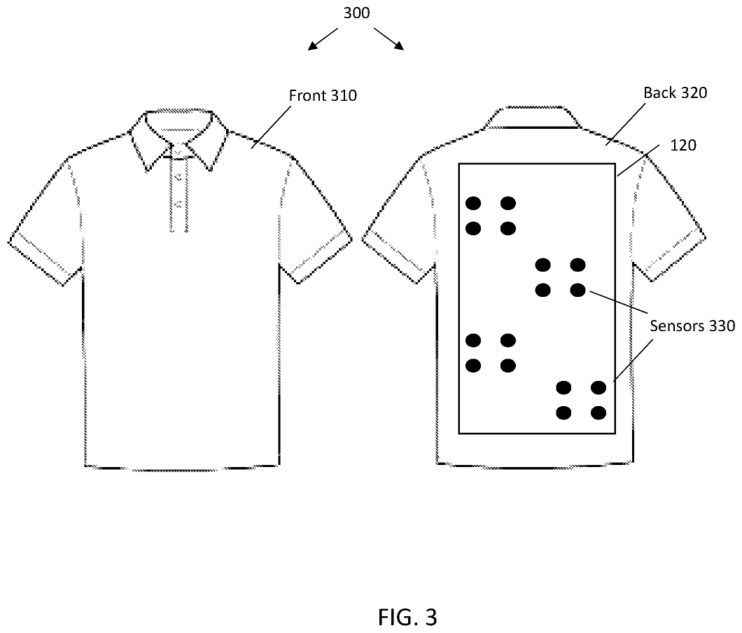

FIG. 3 depicts an example garment according to one or more embodiments. The depicted ECG garment 300 includes various parts, including a front portion 310, a back portion 320, among other portions such as collar, sleeves etc. The front portion 310 is in contact with a front of the user (chest, stomach etc.). The back portion 320 is in contact with the back of the user. The sleeves are in contact with the arms of the user. As described earlier, in existing solutions, the sensors are placed such that they are in contact with the front of the user.

The ECG garment 300 depicted in FIG. 3, includes the panel 120 on the back portion 320. In one or more embodiments of the present invention, the back portion 320 is itself the panel 120. The sensors 215 from the sensor set 210 of the panel 120 are, accordingly, in contact with the back of the user. It is understood that the positions of the sensors 215 in the back portion 320 of the garment 300 shown in FIG. 3 are exemplary, and that in one or more embodiments, the sensors 215 can be positioned differently. Further, it is understood that although a shirt is depicted in FIG. 3, the ECG garment 300 can be any other piece of clothing, such as vest, apron, gown, undergarment, blanket, sheet, jacket, sweater etc. In any type of ECG garment 300, the panel 120 is integrated such that the electrodes of the sensors 215 are in contact with the user's back. Placing the sensors 215 in the back portion 320 ensures that the contact between the sensors 215 and the user's body stays consistent compared to the sensors being in the front.

However, data acquired from the sensors 215 on the back portion 320 can be different from the measurements collected by sensors placed on the front portion 310. Accordingly, referring back to FIG. 2, the panel 120 includes a translator 220 that adjusts the data acquired from the back portion 320. In one or more examples, the translator 220 is part of a controller 230 that is included in the panel 120. Alternatively, or in addition, the translator 220 is a separate module that is coupled with the controller 230. The translator 220 includes one or more electronic circuits, such as a memory device, a processor, and the like. In addition, the translator 220 can include one or more computer executable instructions that are executable by the processor and/or by the controller 230. Alternatively, or in addition, the translator 220 is a module that is included in the data analyzer 140.

In one or more examples, the translator 220 includes or has access to a conversion model to adjust the data acquired from the back portion 320 to be equivalent to data being acquired from the front portion 310. Such a translation facilitates using the translated data in the later parts of the workflow, such as for displaying the data as an ECG for a medical personnel, or for being analyzed to detect an MI event, or any other cardiac event that is being triaged.

Traditional textile technologies like printing, embroidery and knitting techniques are used in developing the panel 120 to provide the user/patient with the most organic user experience. Unlike any other medical device like chest belt or bracelet, the panel 120 is stretchable, washable, comfortable and lightweight in nature.

The electrodes of the sensors 215 are attached to respective insulated leads and seamlessly integrated with the fabric of the panel 120. The leads are connected to an electronic soft board that transfers the collected data to the data analyzer 140 for further analyses. The data analyzer 140 can use artificial intelligence to analyze the data. For example, the data analyzer 140 receives continuously streamed data from the panel 120 that allows one or more analysis models to be trained for identifying patterns and/or changes in heart function. Upon detection of specific events, the data analyzer 140 sends any data that is associated with the event is to on-call cardiologists for early assessment, in one or more examples. The data analyzer 140 and the analysis models that are used can be cloud based in one or more examples. In one or more embodiments of the present invention, the panel 120 can send measurements from the sensors 215 to the data analyzer 140 in a wireless manner, for example, using the Internet, using near field communication (e.g., BLUETOOTH®, BLE®, etc.). Alternatively, or in addition, the panel 120 can send the measurements to the data analyzer 140 via a wired connection.

Using the AI enabled detection, the data analyzer 140 alerts an on-call cardiologist with these data points via a triage platform, that exchanges recorded data with the cardiologist when a critical heart event is detected. This enables the cardiologist to proactively intervene within minutes rather than hours (as per current procedures), affording a nearly instant, life-saving decision and patient notification to seek immediate treatment.

The technical solutions described herein accordingly facilitate a smart wear, such as the garment 300, that serves as a reliable vehicle for providing critical data to the health care providers. Alternatively, or in addition, the panel 120 can be incorporated in a sheet on which the user can be laid down, and effectively drives towards:
Reducing invasive interventions
Reducing hospital costs and
Saving more lives through early detection

The ECG garment can be used by any user, and particularly for the demographic encompassing senior men and women of age 55 years and above, who have a history of heart event occurrence, and/or are risk stratified towards susceptibility of a heart event, such as high cholesterol levels, diabetes, and hypertension. The technical solutions described herein have various advantages including, but not limited to the following:
Deaths due to heart attacks can be prevented by earlier intervention, as compared to current emergency procedure (˜3 hours).
Smart garment organically integrates without lifestyle interruption.
Consistent reliability and quality of electrode contacts are improved by introducing strategic positioning and elastic innovations in fabric.

Additionally, the technical solutions described herein facilitate the panel 120 to be used in an in-patient hospital setting form factor—a strap vest, that is re-purposed between critical care patients. Further, the panel 120 can be used while the user is being transported, for example, via an ambulance or using any other transportation, in other forms, such as the sheet used on a bed, or stretcher that the user is laid on. The ECG garment 300 can include a predetermined number of electrodes, such as 10 electrodes, that pick up signals from the body that can be represented graphically for detecting any abnormality in heart function. A technical challenge with these electrodes is ensuring contact of the electrodes with the body, particularly when a user is sleeping, in motion (such as during an activity), and the like.

By using the panel 120, the user can be monitored through the different states. For example, the panel 120 can be placed on a sheet while the user is sleeping/lying down on the sheet. The panel 120, which can be detached from the sheet in one or more embodiments of the present invention, can then be attached to another garment 300, for example, a gown, or vest, that the user wears. The panel 120 can be, in this manner, moved from one garment to another, depending on the user's medical needs.

Further yet, to address the inconsistent physical contact of the sensors with the user's body, an electrode is specially fashioned in the panel 120 with a riser that elevates the electrode from the rest of the garment 300. This enhances the consistency of body contact without the need for very tight fitting clothes. Further, this reduces the relative motion between the body and the electrodes which greatly reduces ECG signal to noise ratio. Depending on the user demographic, elasticity can be introduced at strategic sections of the garment 300 to further improve the quality of electrode contact with the body. Traditional textile technologies like printing, embroidery and knitting techniques are used in developing this garment 300 to provide the user/patient with the most organic user experience. Unlike any other medical device like chest belt or bracelet, the garment 300 is stretchable, washable, comfortable and lightweight in nature.

Accordingly, the garment 300 includes one or more sensors 215 for measuring cardiac data. The sensors 215 are made part of the garment 300 using a detachable panel 120, or in one or more embodiments of the present invention, are integrated in the garment 300 itself. The panel 120 can be attached/detached using adhesive, Velcro, clips, buttons, stitches, glue, or any other techniques. An electrode of each of the sensors 215 is located on a riser to elevate the electrode from the rest of the garment 300. A first portion of the garment 300 has a different elasticity than a second portion of the garment 300, to facilitate different portions of a user's body to stay in contact with the electrodes in a consistent manner as the user moves freely. For example, the first portion includes an electrode and the second portion does not include an electrode, and hence, the difference in elasticity of the two portions. The electrode is embedded into the garment using at least one of printing, embroidery and knitting.

In one or more embodiments of the present invention, the panel 120 can be associated with a specific user. For example, the panel 120 has a unique panel-identifier associated with the panel 120. For example, the panel-identifier can be an alpha-numeric value, a hash value, or any other unique sequence of characters. Further, the user has a unique user-identifier, which can also be an alpha-numeric value, a hash value, or any other unique sequence of characters. The panel-identifier can be correlated to the user-identifier. In this manner, when the panel 120 is detached from one garment 300, and attached to another garment 300, the sensor data captured by the panel 120 can be continuously tracked for the same user. in one or more embodiments of the present invention, the user identifier can be associated with multiple panel identifiers. This facilitates the user to use multiple garments 300 and/or panels 120 to continuously monitor his/her cardiac health.

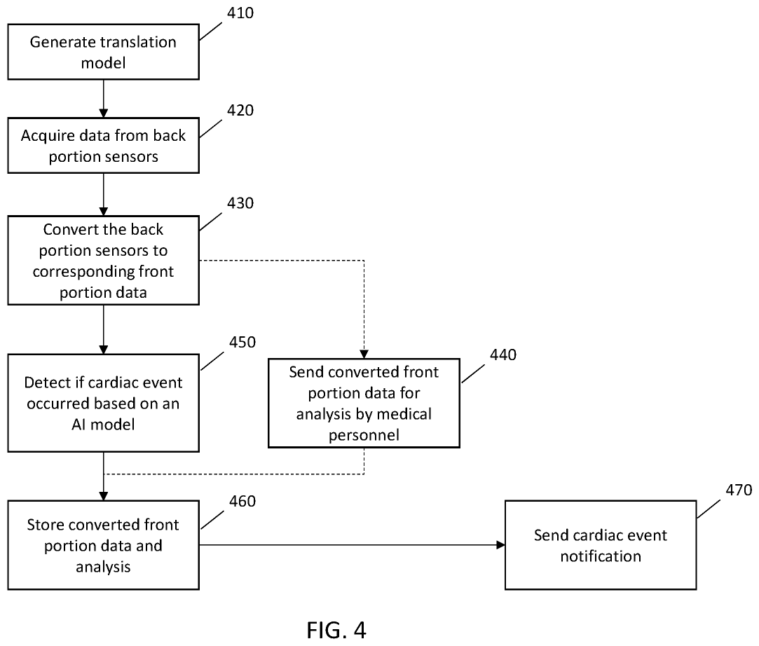

FIG. 4 depicts a flowchart for an example method of acquiring and using sensor data acquired from the back of a user for cardiac triaging according to one or more embodiments. The method includes generating a translation model for converting back portion data, which is data acquired by the sensors 215 from the back portion 320 of the garment 300, to front portion data, at block 410. Here, front portion data refers to ECG data that is (typically) acquired by sensors that are placed in contact with the front of the user, in traditional/existing solutions.

The translation model is generated by correlating the back portion data with the front portion data for multiple users, and in multiple different conditions. The translation model is developed by collecting front portion data and back portion data for multiple users. Further, a relationship between the front portion data and the corresponding back portion data is determined. In one or more examples, a neural network or any other machine learning technique can be used to develop translation model by determining the relationship between the back portion data and the corresponding front portion data.

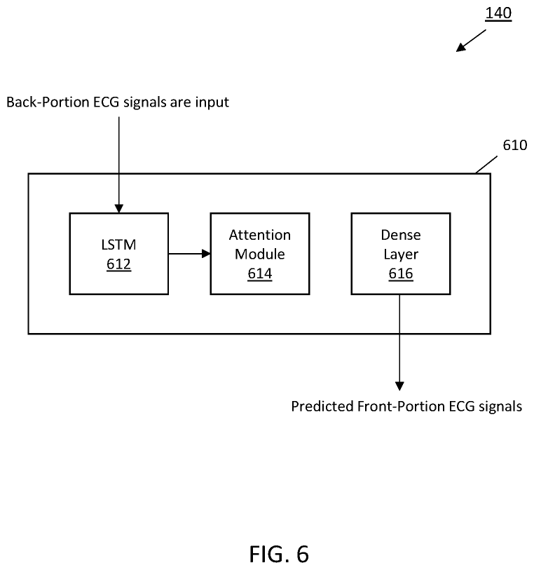

FIG. 6 depicts an example block diagram of a data analyzer according to one or more embodiments of the present invention. The data analyzer 140 generates the translation model using a neural network 610 using an encoder-decoder framework. Such a framework includes an encoder to obtain an embedding of the back ECG signals that are obtained from the back portion of the user. Further, the framework includes a decoder to obtain the translated ECG from the processed back-portion ECG from the encoder. In one or more embodiments of the present invention, a long short-term memory (LSTM) 612, or any other type of recurrent neural network (RNN) is used with an attention module 614 for obtaining the embedding from the back-portion ECG signals. In one or more embodiments of the present invention, the attention module 614 is a self-attention module. The embedding is a representation of the input, in this case the back-portion ECG signals in a format that can be analyzed by further components of the neural network 610. For example, once the embedding is obtained, it is passed to a decoder 616, which can be a fully connected neural network with multiple layers, e.g., a dense layer. The decoder 616 outputs a translated signal based on the embeddings. The translated signal represents a front-portion signal corresponding to the back-portion ECG signal, where front-portion signal is an ECG signal that is obtained by sensors in contact with the front of the user's torso. While training the neural network 610, a loss function, such as cross-entropy loss function is used in one or more embodiments of the present invention. The loss function facilitates the neural network 610 to adjust one or more parameters, e.g., weights of one or more neurons in the various components described herein, to obtain the translation. After training, the configuration of the one or more parameters of the neural network 610 represents a trained translation model, and the neural network 610 serves as fixed network.

Alternatively, or in addition, the translation model can be a linear/non-linear conversion that adjusts the data acquired by multiple sensors 215 from the back portion of the garment 300 into the corresponding front portion data. The translation model can include multiple equations for such adjustment, each equation associated with a particular sensor/data-stream that is used for the analysis.

For example, if the analysis of the data includes using an AI model using 10 streams of data, the translation model translates the back portion data to create 10 streams of data for such analysis. It is to be noted that the back portion data may include different number of data streams (sensors) than those used by the AI model. For example, the sensor-set 210 may include 12 sensors, 15 sensors, 8 sensors, or any other number of sensors. The data streams from such sensors 215 on the back portion 320 are converted into the number of streams (e.g. 10 streams) of data that the AI model of the data analyzer 140 uses for detecting any abnormality in the heart condition.

Referring back to the flowchart in FIG. 4, the method further includes acquiring back portion data for the user, at block 420. The back portion data is acquired using the sensors 215 that are in contact with the user's back through the panel 120. In one or more embodiments of the present invention, the sensors may have accumulated data over a period of time, since the last time the data from the panel 120 was transferred to the data analyzer 140. All of the data from the previous such reading is transferred to the data analyzer 140. As noted earlier, the data can be transferred wirelessly or in a wired manner. For example, one or more lead connectors on the panel 120 are connected to the data analyzer 140, for example via a port such as a USB port, or any other such port.

Further, the method includes using the translation model to convert the acquired data into corresponding front portion data for the data analyzer 140, at block 430. The front portion data is then used to generate and display an ECG waveform to one or more medical personnel for analysis and visualization, at block 440. The data analyzer 140 can generate the ECG waveform using the front portion data according to known techniques because the front portion data is in the predetermined format with which the data analyzer 140 typically operates.

In one or more examples, the data analyzer 140 uses an AI model to detect if a cardiac event, such as an MI has occurred, at block 450. In one or more embodiments of the present invention, the AI model can be fed the back portion data for the analysis. In such cases, the cardia event detection can occur prior to the data translation. Alternatively, in one or more embodiments of the present invention, the front portion data is used for the cardiac event detection by the AI model.

Further yet, the converted data and corresponding analysis is stored in one or more storage devices associated with the data analyzer 140, at block 460. In one or more embodiments of the present invention, a notification is sent if a cardiac event is detected, at block 470.

Accordingly, technical solutions herein facilitate positioning one or more sensors for measuring and accumulating a user's biometric data. Throughout the description, examples are provided for measuring and accumulating cardiac data. However, it is understood that in one or more embodiments of the present invention, other biometric data can be measured and accumulated. For example, a breathing pattern, an oxygen level, a blood pressure, a blood glucose level, or any other such biometric measurements can be captured.

Particularly for the cardiac data capture, the technical solutions herein facilitate positioning one or more sensors on the posterior of a human torso to capture the cardiac data, such as ECG data using only the sensors on the posterior side of the human torso. No sensors are placed on the front of the human torso. Positioning the sensors in this manner improves the contact of the electrodes of the sensors with the torso even when the user is moving (or being moved). For example, when the user is being transported (e.g., in an ambulance), sleeping on a bed, sitting, walking, standing, bending, etc. To further improve the positioning, the sensors are incorporated into a panel of a clothing item/garment. In one or more examples, the panel is detachable from the clothing item/garment. The panel can be attached and detached from garments such as sheets, vests, shirts, undergarments, and other clothing items. The data accumulated by the sensors on the panel can be transferred and analyzed to detect any abnormality. The detection of the abnormality can be performed using data analysis on the captured cardiac data. In one or more examples, the cardiac data has to be translated for using typical data analysis techniques that are used for data that is captured from the front of the torso. Alternatively, techniques are described herein to use the data captured from the posterior of the torso to perform the data analysis and detect cardiac event(s) for the user.

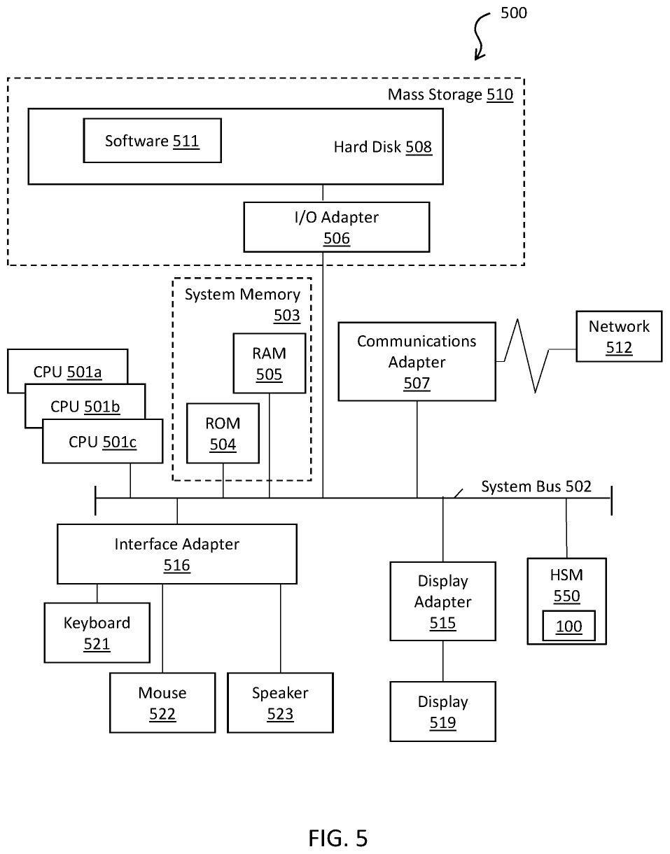

Turning now to FIG. 5, a computer system 500 is generally shown in accordance with an embodiment. The computer system 500 can be an electronic, computer framework comprising and/or employing any number and combination of computing devices and networks utilizing various communication technologies, as described herein. The computer system 500 can be easily scalable, extensible, and modular, with the ability to change to different services or reconfigure some features independently of others. The computer system 500 may be, for example, a server, desktop computer, laptop computer, tablet computer, or smartphone. In some examples, computer system 500 may be a cloud computing node.

The computer system 500 operate as the data analyzer 140. In one or more examples, the computer system 500 receives the sensor data from the panel 120. The computer system 500 can also include the translator 220 in one or more examples. Further yet, the data analyzer 140 can send a notification to the user or a medical personnel upon detection of a cardiac event.

Computer system 500 may be described in the general context of computer system executable instructions, such as program modules, being executed by a computer system. Generally, program modules may include routines, programs, objects, components, logic, data structures, and so on that perform particular tasks or implement particular abstract data types. Computer system 500 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, the computer system 500 has one or more central processing units (CPU(s)) 501a, 501b, 501c, etc. (collectively or generically referred to as processor(s) 501). The processors 501 can be a single-core processor, multi-core processor, computing cluster, or any number of other configurations. The processors 501, also referred to as processing circuits, are coupled via a system bus 502 to a system memory 503 and various other components. The system memory 503 can include a read only memory (ROM) 504 and a random access memory (RAM) 505. The ROM 504 is coupled to the system bus 502 and may include a basic input/output system (BIOS), which controls certain basic functions of the computer system 500. The RAM is read-write memory coupled to the system bus 502 for use by the processors 501. The system memory 503 provides temporary memory space for operations of said instructions during operation. The system memory 503 can include random access memory (RAM), read only memory, flash memory, or any other suitable memory systems.

The computer system 500 comprises an input/output (I/O) adapter 506 and a communications adapter 507 coupled to the system bus 502. The I/O adapter 506 may be a small computer system interface (SCSI) adapter that communicates with a hard disk 508 and/or any other similar component. The I/O adapter 506 and the hard disk 508 are collectively referred to herein as a mass storage 510.

Software 511 for execution on the computer system 500 may be stored in the mass storage 510. The mass storage 510 is an example of a tangible storage medium readable by the processors 501, where the software 511 is stored as instructions for execution by the processors 501 to cause the computer system 500 to operate, such as is described herein below with respect to the various Figures. Examples of computer program product and the execution of such instruction is discussed herein in more detail. The communications adapter 507 interconnects the system bus 502 with a network 512, which may be an outside network, enabling the computer system 500 to communicate with other such systems. In one embodiment, a portion of the system memory 503 and the mass storage 510 collectively store an operating system, which may be any appropriate operating system, such as the z/OS or AIX operating system from IBM Corporation, to coordinate the functions of the various components shown in FIG. 5.

Additional input/output devices are shown as connected to the system bus 502 via a display adapter 515 and an interface adapter 516 and. In one embodiment, the adapters 506, 507, 515, and 516 may be connected to one or more I/O buses that are connected to the system bus 502 via an intermediate bus bridge (not shown). A display 519 (e.g., a screen or a display monitor) is connected to the system bus 502 by a display adapter 515, which may include a graphics controller to improve the performance of graphics intensive applications and a video controller. A keyboard 521, a mouse 522, a speaker 523, etc. can be interconnected to the system bus 502 via the interface adapter 516, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 5, the computer system 500 includes processing capability in the form of the processors 501, and, storage capability including the system memory 503 and the mass storage 510, input means such as the keyboard 521 and the mouse 522, and output capability including the speaker 523 and the display 519.

In some embodiments, the communications adapter 507 can transmit data using any suitable interface or protocol, such as the internet small computer system interface, among others. The network 512 may be a cellular network, a radio network, a wide area network (WAN), a local area network (LAN), or the Internet, among others. An external computing device may connect to the computer system 500 through the network 512. In some examples, an external computing device may be an external webserver or a cloud computing node.

It is to be understood that the block diagram of FIG. 5 is not intended to indicate that the computer system 500 is to include all of the components shown in FIG. 5. Rather, the computer system 500 can include any appropriate fewer or additional components not illustrated in FIG. 5 (e.g., additional memory components, embedded controllers, modules, additional network interfaces, etc.). Further, the embodiments described herein with respect to computer system 500 may be implemented with any appropriate logic, wherein the logic, as referred to herein, can include any suitable hardware (e.g., a processor, an embedded controller, or an application specific integrated circuit, among others), software (e.g., an application, among others), firmware, or any suitable combination of hardware, software, and firmware, in various embodiments.

Although specific embodiments of the invention have been described, one of ordinary skill in the art will recognize that numerous other modifications and alternative embodiments are within the scope of the invention. For example, any of the functionality and/or processing capabilities described with respect to a particular system, system component, device, or device component may be performed by any other system, device, or component. Further, while various illustrative implementations and architectures have been described in accordance with embodiments of the invention, one of ordinary skill in the art will appreciate that numerous other modifications to the illustrative implementations and architectures described herein are also within the scope of this invention. In addition, it should be appreciated that any operation, element, component, data, or the like described herein as being based on another operation, element, component, data, or the like may be additionally based on one or more other operations, elements, components, data, or the like. Accordingly, the phrase “based on,” or variants thereof, should be interpreted as “based at least in part on.”

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A system for monitoring cardiac health of a human, the system comprises:
a garment comprising:
a front portion that contacts the anterior of a human torso; and
a back portion that contacts the posterior of the human torso, wherein the back portion comprises one or more cardiac sensors.

2. The system of claim 1, wherein an electrocardiogram is generated using sensor data that is captured only by the one or more cardiac sensors on the posterior of the human torso.

3. The system of claim 2, further comprising a translator that converts the sensor data that is acquired from the posterior of the human torso to corresponding front portion data, wherein the front portion data represents sensor data captured from the anterior of the human torso, wherein the translation is performed prior to generation of the electrocardiogram.

4. The system of claim 1, further comprising:
a data analyzer that comprises one or more processing units and a memory, the one or more processing units configured to:
receive sensor data that is captured by the one or more cardiac sensors;
analyze the sensor data using a neural network; and
detect a cardiac anomaly in the sensor data based on the analysis.

5. The system of claim 4, further comprising a translator that converts the sensor data that is acquired from the posterior of the human torso to corresponding front portion data, wherein the front portion data represents sensor data captured from the anterior of the human torso, wherein the translation is performed prior to analysis of the sensor data, wherein the front portion data is used for the analysis.

6. The system of claim 4, wherein a notification is sent for receipt by a medical personnel in response to the cardiac anomaly being detected.

7. The system of claim 1, wherein the garment is a first garment, and wherein the one or more sensors are part of a panel that is detachable from the first garment and, the panel is further attachable to a second garment for continuous capturing of cardiac sensor data of the human.

8. A method of monitoring cardiac health of a human, the method comprising:
measuring back portion cardiac data using one or more sensors that are all in contact with a posterior of a human torso;
detecting a cardiac anomaly using machine learning based on the back portion cardiac data.

9. The method of claim 8 further comprising, converting the back portion cardiac data to front portion cardiac data using a translation model, wherein the front portion cardiac data is used for detecting the cardiac anomaly.

10. The method of claim 9 further comprising, generating an electrocardiogram using the front portion cardiac data.

11. The method of claim 8 further comprising, notifying a medical personnel in response to detecting the cardiac anomaly.

12. The method of claim 8, wherein the one or more sensors are positioned using a panel that is incorporated into a garment that is in contact with the human torso.

13. The method of claim 12, wherein the garment is a first garment, and the method further comprising:
in response to a second garment being brought in contact with the posterior of the human torso:
detaching the panel from the first garment; and
attaching the panel to the second garment.

14. The method of claim 12, wherein the garment is one from a group of clothing items comprising sheet, vest, gown, apron, shirt, undergarment, strap, and outerwear.

15. A garment comprising:
a front portion that contacts the anterior of a human torso of the user;
a back portion that contacts the posterior of the human torso; and
a panel that is incorporated with the back portion, the panel comprises a plurality of cardiac sensors to monitor cardiac health of the user.

16. The garment of claim 15, wherein an electrocardiogram is generated using sensor data that is captured by the plurality of cardiac sensors on the posterior of the human torso.

17. The garment of claim 16, wherein the sensor data that is acquired from the posterior of the human torso is converted, by a translator, to corresponding front portion data, wherein the front portion data represents sensor data captured from the anterior of the human torso, wherein the translation is performed prior to generation of the electrocardiogram.

18. The garment of claim 17, wherein the translator is part of the panel.

19. The garment of claim 17, wherein the translator is part of a data analyzer that receives the sensor data from the panel.

20. The garment of claim 19, wherein the data analyzer detects a cardiac anomaly by analyzing the sensor data using machine learning.
